(19) United States
(12) Patent Application Publication
(10) Pub. No.: US 2007/0093539 A1
(43) Pub. Date: Apr. 26, 2007

(54) N-hydroxy-4-(3-pheyl-5-methyl-isoxazole-4-yl)-benzenesulfonate solvates

(76) Inventors: Janos Fischer, Budapest (HU); Tamas Fodor, Budapest (HU); Egon Karpati, Budapest (HU); Istvanne Kis-Varga, Kakucs (HU); Sandor Levai, Bitorbagy (HU); Peter Erdelyi, Budapest (HU); Maria Zajerne Balazs, Budapest (HU); Aniko Gere, Budapest (HU)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/559,702

(22) PCT Filed: Jul. 16, 2004
(86) PCT No.: PCT/HU04/00077

§ 371 Date: Dec. 6, 2005

(30) Foreign Application Priority Data
Jul. 16, 2003 (HU) P 0302219

(51) Int. Cl. A61K 31/42; C07D 261/06

(52) U.S. Cl. 514/378; 548/247

(57) ABSTRACT

The invention relates to new N-hydroxy-4-(3-phenyl-5-methyl-isoxazole-4-yl)-benzenesulfonamide solvates of formula (I). 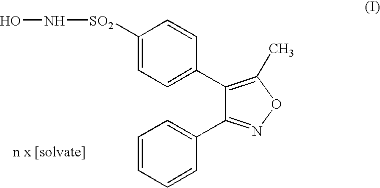 wherein n represents 0 or 1 mol,
[solvate] represents water, C₁-C₄ alcohol, C₁-C₄ alkylester of C₁-C₃ carboxylic acid or dioxane, and the mixture of solvated (wherein n=1) and solvat-free forms (wherein n=0). Furthermore, the invention relates their process for production and use for the treatment of osteoarthritis and rheumatoid arthritis and surgical and primary dysmenorrheal pains, based on anti-inflammatory and analgesic pharmacological model experiments.

The present invention relates to new N-hydroxy-4-(3-phenyl-5-methyl-isoxazole-4-yl)-benzenesulfonamide solvates of formula (I)

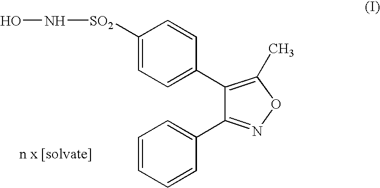

wherein n represents 0 or 1 mol,

[solvate] represents water, C₁-C₄ alcohol, C₁-C₄ alkylester of C₁-C₃ carboxylic acid or dioxane, and the mixture of solvated (wherein n=1) and solvate-free forms (wherein n=0). Furthermore, the present invention relates to their process of production and their use for the treatment of osteoarthritis and rheumatoid arthritis and surgical and primary dysmenorrheal pains, based on anti-inflammatory and analgesic pharmacological model experiments.

It is known that the side effect profiles of selective cyclooxygenase-2 inhibitors are much more favourable than those of the non-steroidal antiinflammatory drugs. It concerns first of all their gastrointestinal activity.

Presently two generations of selective cyclooxygenase-2 inhibitors are known. One of the first cyclooxygenase-2 inhibitors in the market was celecoxib. Celecoxib has high selectivity and decreases the gastrointestinal side effects significantly, but does not eliminate totally.

Valdecoxib, a member of second generation of COX-2 enzime inhibitors has been launched for treatment of osteoarthritis, rheumatoid arthritis and dysmenorrhea pain in 2002. It is known in the literature that the gastrointestinal side effects are shown also in administration of valdecoxib.

It should be taken into account that the selective cyclooxygenase-2 inhibitors have cardiovascular side effect, too.

These facts are shown in a study of another first generation COX-2 inhibitor, rofecoxib. (Vigor-study, Bombardier C, Laine L, Reicin A et al for the VIGOR Study Group. Comparison of upper gastrointestinal toxicity of rofecoxib and naproxen in patients with rheumatoid arthritis. N Engl J Med 343(21): 1520-1528, November 2000.)

The possible causes were discussed in detail in study of D. Mukherjee. (Mukherjee D, Nissen S E, Topol E J. Risk of cardiovascur events associated with selective COX-2 inhibitors. JAMA 2001; 286: 954-959).

In order to solve the above-mentioned problems more potent selective cyclooxygenase-2 inhibitors were researched.

Surprisingly, we have found that N-hydroxy-4-(3-phenyl-5-methyl-isoxazole--4-yl)-benzenesulfonamide solvates of formula (I) (wherein n=1) and solvate-free forms (wherein n=0) or their mixtures have more advantageous effect profile than valdecoxib.

In an article (Josh J. Yuan, Dai-Chang Yang, Ji Y. Zjang, Roy Bible Jr., Aziz Karim és John W. A. Findlay: Drug Metabolism and Disposition Vol. 30 (No.9), 1013-1021 (2002)) it is described that the solvate-free N-hydroxy-4-(3-phenyl-5-methyl-isoxazole-4-yl)-benzenesulfonamide is defecated in urine as metabolite of valdecoxib. The compound was identified by mass spectroscopy as a minor metabolite of valdecoxib, but the preparation, biological and chemical properties of the compound were not reported.

Compounds of general formula (I) should be taken into the group of the selective cyclooxygenase-2 inhibitors because they have considerable selectivity of cyclooxygenase-2 enzime, as it is shown in Table 1. Compounds of general formula (I) in the respect of the main effects (antiinflammation and analgesic), show more favourable properties than valdecoxib, because these give significantly better results in in vivo tests than valdecoxib.

Compounds of general formula (I) have more advantageous profiles in the respect of side effects than valdecoxib: they increased the velocity of the blood-stream, which effect is advantageous in practice of clinical therapy. The painful arthritic and degenerative joint and bone deformations are emerged rather in old age, when diseases of the vascular system are also frequently encountered, which can conduce to disorder of vascular bed of the heart. In this case a therapy used for joint and bone problems can extremely be advantegeous, if it improves significantly the vascular bed of the heart, too.

During the preparation of N-hydroxi-4-(3-phenyl-5-methyl-isoxazole-4-yl)-benzenesulfonamide we have found that the properties of solvated forms more favourable than the properties of amorf compounds because these are crystallized and can be handled easily. The solvates of formula (I) contain 1 mole solvate as inclusion compound (n=1). The solvate may be one mole water, one mole C₁-C₄ alcohol, one mole C₁-C₄ alkylester of C₁-C₃ carboxylic acid or one mole dioxane. The solvates of compounds of general formula (I), where n=1, could loose some of their solvates under the conditions of preparation or isolation. The solvate-free form of compounds of general formula (I) can be formed in vacuum under heating. Ratio of the solvated and solvat free forms can be adjusted with changing the time of the heating.

Starting material of the compounds of general formula (I) was 3-phenyl-4-(4-chlorosulfonyl-phenyl)-5-methyl-isoxazole (II). It was prepared from 3,4-diphenyl-5-methyl-isoxazole (III) by reaction of chloro sulfonic acid. Preparation of compound of formula (III) can be prepared by the following article: P. Bravo, G. Gaudiano, C. Ticozzi: Gazz. Chim. Ital. 102, 395 (1972).

The sulfonation was carried out in inert organic solvent, preferably in water-free dichoromethane, namely the 3,4-diphenyl-5-methyl-isoxazole was reacted with excess of chloro sulfonic acid, preferably with fivefold excess under heating, preferably on boiling point of reaction mixture.

The compound of formula (II) can be coupled to hydroxy-sulfonamides in two different processes.

In case of method a., the chlorosulfonyl derivative was reacted with hydroxylamine in mixture of water-soluble solvent and water. The reaction time was 15 to 45 minutes, preferably 30 minutes. The reaction temperature was 15 to 25° C. The reaction mixture was added to the water, the product was filtered, and washed with water. The crude product was crystallized from the mixture of water and ethanol and the final product was a monohydrate (I, n:1, solvate:H₂0) in a yield of 70%, the purity of 99.8%.(HPLC).

In case of method b., the chlorosulfonyl derivative was reacted with hydroxylamine in mixture of non-water-soluble solvent, preferably ethylacetate and water in presence of phase-transfer catalyst, preferably tetrabutyl ammonium hydrogensulfate. The reaction was carried out in room temperature, the reaction time was 5 to 20 hours. The crude product obtained after the preparation was crystallized, and it was recrystallized from mixture of water and alcohol, preferably from mixture of water and ethanol. The yield was 60%. The solvate of the obtained product was water.

The preparation of solvate-free N-hydroxi-4-(3-phenyl-5-methyl-isoxazole-4-yl)-benzenesulfonamide was carried out by heating of the solvated compounds of general formula (I), preferably heating of the N-hydroxi-4-(3-phenyl-5-methyl-isoxazole-4-yl)-benzenesulfonamide monohydrate. The time of the heating was 20 to 40 minutes, preferably 25 minutes.

In Vitro Studies

The Human recombinant COX-2 and sheep COX-1 activity were determined by spectrophotometric TMPD assay (K. Gierse, S. D. Hauser, D. P. Creely, C. M. Koboldt, S. H. Rangwala, P. C. Isakson and K. Seibert: Expression and selective inhibition of the constitutive and inducible forms of human cyclo-oxygenase Biochem. J. 305: 479-484 (1995)).

Principle of the Measurement

The Human recombinant COX-2 and sheep COX-1 activity were measured by spectrophotometric assay based on oxidation of N,N,N′,N′,-tetramethyl-p-phenylenediamine (TMPD). During the reduction of the prostaglandine G₂ (PGG₂) to prostaglandine endoperoxid H₂ (PGH₂), the TMPD is being oxidated to a colour product, which can be measured by spectrophotometer at 610 nm.

Method:

4 μl of solution of inhibitors in different concentrations was added to the 156 μl of reaction mixture (100 mM sodium phosphate buffer, pH:6.5, 1 μM hematin, 1 mg/ml gelatine). Afterwards 20 μl solution of 50 unit human recombinant COX-2 enzym or 20 μl of 50 unit sheep COX-1 enzym (Cayman Chemical, Ann Arbor, USA, cat no.: 60122/COX-2/, cat. no.: 60100/COX-1/) was added. The incubation mixture: was preincubated for 15 minutes at 25° C. in a spectrophotometric 96-well plate reader (Labsystem iEMS Reader MF). Following this, the mixture of 20 μl 1 mM arachidonic acid and 1 mM TMPD solution were added. It was shaken for 10 second and the absorbance was measured at 610 nm. The results are summarized in Table 1.

TABLE 1

| | Human recombinant COX-2 | Sheep COX-1 |
|---|---|---|
| compound | IC₅₀ (μM) ± S.E.M. | IC₅₀ (μM) ± S.E.M. |
| compound as | 1.1 ± 0.3 | 101.4 ± 12.5 |
| example 1 | | |

In Vivo Studies
1. Carrageenan-Induced Rat Foot Paw Edema Assay

The edema was induced in male Wistar rats (140-150 g) by subcutaneous injection of carrageenan (50 μl of 1% suspension) into the right hind paw. The formed inflammation was measured with plethysmometer (Ugo Basile, type: 7150). The treated paw was placed into the plethysmometer (filled with 0.3% additives in 0.5% saline), the level of the inflammation was detected by the volume of the displaced solution. This volume was compared with the initial preinjection paw volume.
Level of the inflammation (ml)=volume after CA treatment (ml)−volume before CA treatment (ml)
The inflammation induced in treated group was compared to control group (which was given only vehicle).

The sample materials and the solvent were dosed per os via gastro-sonde 1 hour before the CA treatment. The volume of the treated limb was measured at 3h and 5h after CA treatments. The change of the inflammation level was calculated as follows:

$$\% Inhibition of inflammation = \frac{Control group ( ml ) - treated group ( ml )}{control group ( ml )}$$

Wide dose range of valdecoxib (0.1-0.3-1-3 mg/kg) and compound of example 1 were examined (n=6-12 animal/group). The levels of the inflammation inhibition effect of the compounds were determined in % at 4 and 6 hours after the treatment and the ED₃₀ of inflammation inhibition was calculated.

The results: Edema inhibition effect of valdecoxib at 4 hours after the treatment ED₃₀=0.2 mg/kg, at 6 hours after the treatment ED₃₀=0.3 mg/kg.

Edema inhibition effect of compound of example 1 at 4 hours after the treatment ED₃₀=1.8 mg/kg, at 6 hours after the treatment ED₃₀=0.8 mg/kg.

The edema inhibition effects of both compounds were significant, as the results show. The inhibition effect of valdecoxib was higher at 4 hours than of compound of example 1. However, effect profile of compound of example 1 was favourable, because it was more effective at 6 hours than at 4 hours.

The results are summarized in Table 2.

TABLE 2
Inhibition of inflammatory mechanical allodynia in rats induced by
Carrageenan

| | Time after | edema inhibition effect % | | | | | |
|---|---|---|---|---|---|---|---|
| | treatments | Doses (mg/kg p.o.) | | | | | ED₃₀ |
| Compounds | (hours) | 0.1 | 0.3 | 1.0 | 3.0 | 10.0 | mg/kg |
| valdecoxib | 4 | 29.4 | 34.1 | 40.1 | 47.7 | — | 0.2 |
| | 6 | 25.8 | 27.3 | 37.8 | 45.8 | — | 0.3 |
| Compound of | 4 | — | 19.5 | 25.2 | 33.9 | 40.7 | 1.8 |
| example 1 | 6 | — | 17.5 | 37.2 | 40.4 | 59.2 | 0.8 |

The-threshold of pain of the animals was measured by von Frey apparatus (IITC, type: 1601 C). The stimulus threshold was measured by continously increased power on the central region of the plantar surface. The values were registered in the times of the pick up or raises. During each measurment the threshold was determined at least thrice and the avarage was calculated from the peak values.

Male Sprague-Dawley rats (weighing 250-300 g) were used (n=5-6/group). 100 μl of saline solution of carrageenant (CA) was injected to the middle of the paw. The stimulus threshold was measured after it, and the treatment was completed with gastro-probe per os. The effects of the materials were measured in 30, 60, 90, 120 minutes after the treatments. The effects were compared for control group treatment with vehicle (solution of 2% Tween-80).

The effect was calculated as follows:

$$analgesic effect \% = \frac{\begin{matrix}threshold of the treated grp . ( {t}_{x} ) - \\ threshold of the cont . grp . ( {t}_{x} )\end{matrix}}{\begin{matrix}threshold of the treated grp after CA ( {t}_{0} ) - \\ threshold of the cont . grp ( {t}_{x} )\end{matrix}}$$

where tₓ=30, 60, 90, 120 min

In the acute pain model the analgesic effects of the valdecoxib and compound of example 1 were considerable with dose of 30 mg/kg p.o. once.

The inhibition effect of the valdecoxib was a little bit higher (5-10%) than effect of compound example 1, however this difference was not statistically significant. The results are shown in Table 3.

TABLE 3

| | | Analgesic effect in % | | | |
|---|---|---|---|---|---|
| | Dose | after p.o. treatment | | | |
| compounds | (mg/kg p.o.) | 30 min. | 1 hour | 1.5 hour | 2 hours |
| Valdecoxib | 30 | 69.2 | 60.0 | 57.7 | 47.8 |
| compound | 30 | 61.6 | 57.1 | 44.5 | 41.5 |
| example 1 | | | | | |

3. Carrageenan and Kaolin Induced Monoarthritis Modell in Rats (Incapacitance Test)

The Incapacitance tester is an apparatus for measure the changes of the functional parameters induced of the pain, which can register the bearing on the hind limb, the amount of the moving and the changing of the centre of gravity.

Knee-joint of the right-back limb was treated with solution of 100 μl 2% carrageenan and kaolin. During 3-4 hours after the treatment arthritis was emerged in the capsular ligament of the treated limb. This inflammation still exists at 24 hours after the treatment. Because of the pain the animals coddles the treated limbs, thy weign on it less. Change of weight load is measurable with Incapacitance tester device in grams.

The incapacitance was calculated as follows:

$$Incapacitance ( IC \% ) = \frac{Left limb ( g ) - Right limb ( g )}{Left limb ( g ) + Right limb ( g )} \times 100$$

Analgesic-antiinflammantory compounds could increase the stimulus threshold of the knee-joint, and consequently to improve the functional parameters of the limb. Measure of this can be counted by the decrease of the loading of left leg i.e. in terms of percentage of reversal.

$$\% Reversal = 100 \frac{Incapacitance \% of the left limb after treatment}{Incapacitance \% of the left limb before treatment} \times 100$$

The Incapacitance induced by administration of irritants in left paw was measured at 4 hour after the injection. Afterwards the animals (n=24-32/group) were. orally treated with dose of 10 mg/kg valdecoxib and test compounds. Measurements were done at 1 and 2 hours after the treatments. Analgesic effects of both compounds were significant at 1 hour and were increased to next hour. The effects of compound of example 1 were higher with 20% in both points of measurement than effects of valdecoxib.

The results are summarized in Table 4.

TABLE 4

| | Dose | | |
|---|---|---|---|
| | mg/kg | Analgesic effect (% reversal) after p.o. treatment | |
| Compounds | p.o. | 1 hour | 2 hours |
| valdecoxib | 10 | 52.1 | 62.4 |
| compound of | 10 | 63.2 | 76.9 |
| example 1 | | | |

4. Carragenaan Induced Inflammatory Hyperalgesia Model in Rats (Randall-Selitto's Method)

Edema was induced by injection of Carrageenan (CA) into plantar surface of right hind paw. Male SPRD rats (weighing 140-190 g) were used n=6-8/group). Then the mechanical pain threshold of the inflamed hind paw was determined with an analgesimeter ((Ugo Basile, type: 37215).

This assay monitors the decrease of the pain threshold, and the time depending changing of the pain by mechanical pain stimulus. Analgesics can increase the pain threshold of the inflamed hinds and this effect is in terms of percentage of reversal.

Untreated right hind paw was compressed with a progressively increasing pressure. The pressure was recorded (in grams) when the animal first vocalized or made a vigorous attempt to remove the paw. It was determined to baseline threshold of the untreated paw. (average: 80-110 g). After it the animals were treated with carrageenan. After the treatment the edema and the threshold were checked in a given times. The CA induced threshold decrease was observed at 3 hours after the injection. (Average of inflammation induced pain threshold was 20-25 g, this means 65-80% decrease related to the baseline.)

Acute Model:

1 hour after the CA injection (100 μl of 2% solution) the animals were treated with test compounds and valdecoxib (10-10 mg/kg p.o.). Change in the threshold was measured at 2 hours after the administration.

Chronic Model:

The chronic phase of the inflammantion and the decreasing of the threshold were induced by higher dose of CA. Inflammation induced threshold decreasing was measured 24 hours after the CA injection (150 μl of 2% solution). ) After it the animals were treated with test compounds and valdecoxib (30-30 mg/kg p.o.). Change in the threshold was checked at 1 h, 2 h, and 3 h following drug administration. In both model used control groups were treated orally only with solvents at the time of treatments. In both protocol the effects of the test compounds were calculated in percentage reversal of mechanical hiperalgesia:

$$\% Reversal = \frac{Av . of Treated {grp}_{Txh} ( g ) - Av . of Control {grp}_{T 3 h / T 24 h} ( g )}{Baseline of Control {grp}_{T 0 h} ( g ) - Av . of Control {grp}_{T 3 h / T 24 h} ( g )} \times 100$$

T₃ₕ: In acute model, the threshold in the control group at 3 h after the CA injection (in gram)
T₂₄ₕ: In chronic model, the threshold in the control group at 24 h after the CA injection (in gram)
T₀ₕ: The threshold before the CA injection (in gram)
T_{Xh} In acute model, at 3h after the CA injection
T_{Xh}=In chronic model, at 25 h, 26 h, 27 h after the CA injection

The valdecoxib and the test compound produced significant antihyperalgesic effects in acute and chronic models. In the chronic model, at all three measure times, the compound of example 1 was more effective then valdecoxib. Results of acute and chronic models are summarized in Table 5 and 6, respectively.

TABLE 5

| | | Analgesic effect in % | | |
|---|---|---|---|---|
| | Dose | after p.o. treatment | | |
| Acut model | (mg/kg p.o.) | 2 hours | 3 hours | 4 hours |
| valdecoxib | 10 | 50.5 | 59.9 | 33.2 |
| compound of | 10 | 64.6 | 40.7 | 12.3 |
| example 1 | | | | |

TABLE 6

| | | Analgesic effect in % | | |
|---|---|---|---|---|
| | Dose | after p.o. treatment | | |
| Chronic model | (mg/kg p.o.) | 1 hour | 2 hours | 3 hours |
| valdecoxib | 30 | 24.2 | 36.9 | 19.9 |
| Compound of | 30 | 57.8 | 63.9 | 42.0 |
| example 1 | | | | |

5. Cardiac Effects on the Isolated Rabbit Heart

New Zealand white rabbits weighing 1.5-2 kg were used. The animals were exsanguinated and after thoracotomy the hearts were excised and mounted on a Langendorff-type perfusion apparatus. The hearts were perfused via the aorta with oxigenated, thermostated (37° C.) Krebs solution. A constant perfusion pressure of 80 cmH₂O was applied. The test compounds were dissolved in the perfusion solution to obtain the requested concentrations.

The basic value of coronary flow was determined. Afterwards the small amount of compounds were added to perfusion liquid and the perfusion was measured for 30 minutes at every 10 minutes. Afterwards 30 minutes perfusion was performed without compound and the measure was repeated with the medium and high amount of compounds.

The effects of same concentration of valdecoxib and compound of example 1 were investigated in 4-4 hearts (1, 3 and 10 μM). Valdecoxib had no effect in either concentration. The compound of example 1 had positive effect and the results are summarized in Table 7. It can clearly be shown that the coronary flow was increased dose dependently. This effect is advantageous in practice of clinical therapy, because the painful arthritic and degenerative joint and bone deformations are emerged rather in old age, when diseases of the vascular system are also frequently encountered, which can conduce to disorder of vascular bed of the heart. In this case a therapy used for joint and bone problems can extremely be advantageous, if it improves significantly the vascular bed of the heart, too.

TABLE 7

| Time of the | | | |
|---|---|---|---|
| treatments | concentration in μM | | |
| (minutes) | 1 | 3 | 10 |
| Basic | 34.3 ± 4.4 | 30.0 ± 3.2 | 28.0 ± 1.4 |
| 10% changing | 40.0 ± 3.3 | 37.3 ± 2.3 | 45.8 ± 2.5 |
| | 16.6 | 24.3 | 63.6 |
| 20% | 39.0 ± 3.0 | 37.5 ± 1.7 | 48.3 ± 4.8 |
| changing | 13.7 | 25.0 | 72.5 |
| 30% | 37.5 ± 2.4 | 38.8 ± 1.3 | 49.0 ± 4.8 |
| changing | 9.3 | 26.0 | 75.0 |

The results of biological studies show the followings:
the compounds of general formula (I) have significant COX-2 enzyme selectivity based on in vitro studies,
the effects of the compounds of general formula (I) are higher than effect of valdecoxib as the in vivo test results show,
the compounds of general formula (I) increase the coronary flow.

Implementation of the present invention is shown by the following examples, without any limitaton to them.

The NMR studies were performed in a Varian spectrometer (300 MHz). The HPLC studies were carried out by a Merck-Hitachi-Lachrom equipment.

### EXAMPLE 1

### N-hydroxi-4-(3-phenyl-5-methyl-isoxazole-4-yl)-benzenesulfonamide monohydrate

A.

6.88 g (0.099 mol) hydroxylamine-hidrochloride was suspended in 50 ml dioxane, cooled to +10 C.° and was added solution of 8.1 g (0.099 mole) sodium acetate in 25 ml water. Solution of 11 g (0.033 mole) 3-phenyl-4-(4-chloro-sulfonyl-phenyl)-5-methyl-isoxazole in 50 ml dioxane was added during 30 minutes. The mixture was stirred for 30 minutes and was added to 500 ml of water and the suspension was shaken for 2 hours. The crude product was dissolved in ethyl acetate (200 ml) and the solution was extracted with 5% aqueous solution of ethylenediaminetetraacetic acid disodium salt (40 ml), then with water (40 ml) and finally with brine (20 ml). The solution was evaporated in vacuo. The residue was dissolved in ethanol (90 ml), decolorized by activated carbon (1 g), filtered and water (270 ml) containing ascorbic acid (3 g) was added to the solution at 60 C.°. The solution was cooled (+5 C.°) and the precipitated product was filtered, washed with water and dried to afford the title compound (7.8 g; 68%; mp: 95-110 C.°) ¹H NMR(DMSd₆, 30 C.°, δ_{TMS}: 0.00 ppm): 2.49 s (3H); 7.33-7.52 m (7H); 8.82-7.88 m (2H); 9.67 s (2H). The purity was 99.9% by HPLC.

B. 5.4 g (0.016 mol) of 3-phenyl-4-(4-chlorosulfonyl-phenyl)-5-methyl-isoxazole was dissolved in 65 ml of ethyl acetate. 2.3 ml (0,035 mol) of 50% aqueous solution of hydroxylamine and 0.3 g of tetrabutylammonium hydrogensulfate in water (65 ml). The reaction mixture was stirred at room temperature for 8-20 hours. Ethyl acetate (150 ml) and water (150 ml) were added to the reaction mixture. The organic phase was separated and dried over sodium sulfate, then the solution was evaporated under reduced pressure. The residue (4.9 g) was dissolved in 70 ml of ethanol and after decolorization by activated carbon the solution was filtered. Water (210 ml) was added to the solution and the crystalline product was filtered, washed with water and dried. Yield 3.0 g (54%).

### EXAMPLE 2

### N-hydroxi-4-(3-phenyl-5-methyl-isoxazole-4-yl)-benzenesulfonamide mono-ethyl acetate solvate

6.88 g (0.099 mol) hydroxylamine-hydrochloride was suspended in 50 ml dioxane, cooled to +10 C.° and was added solution of 8.1 g (0.099 mole) sodium acetate in 25 ml water. Solution of 11 g (0.033 mole) 3-phenyl-4-(4-chloro-sulfonyl-phenyl)-5-methyl-isoxazole in 50 ml dioxane was added over a period of 30 minutes. The mixture was stirred for 30 minutes and was added to 600 ml of water and the suspension was stirred for 2 hours. The suspension was filtered and washed 2 times with 100 ml of water. The precipitate was disolved in 300 ml of ethyl acetate, extracted three times with 50 ml water. The organic solution was dried with 5 g of anhydrous magnesium sulfate. After filtration of the magnesium sulfate the solution was evaporated to 80 ml under reduced pressure (40 mbar), while the product is crystallized. This suspension was stirred for 2 hour at −5 C.°, and washed with 10 ml of cooled (−10 C.°) ethyl-acetate. After the drying gave 8.5 g (60%) of the title compound (mp: 96-100 C.°, decomposition at 108 C.°) The purity was 99.9% by HPLC.

### EXAMPLE 3

### N-hydroxy-4-(3-phenyl-5-methyl-isoxazole-4-yl)-benzenesulfonamide mono-2-propanole solvate

4 g of N-hydroxy-4-(3-phenyl-5-methyl-isoxazole-4-iy)-benzene-sulfonamide mono-ethyl acetate solvate was dissolved in 20 ml of 2-propanol at 45 C.°. The heating was stopped and the title compound was precipitated. The suspension was stirred for 2 hours at 0 C.° and filtered to give the title compound (3.6 g; 96%; mp.: 100-118 C.°, decomposition at 123 C.°).

### EXAMPLE 4

### N-hydroxy-4-(3-phenyl-5-methyl-isoxazole-4-yl)-benzenesulfonamide mono-dioxane solvate

100 mg of the title compound of example 3 was dissolved in 10 ml of dioxane, heated to 40 C.° and was added dropwise 10 ml of water. The product was precipitated in crystallized form at 20 C.°. The suspension was stirred at 2 hours, filtered and the product was dried at 25 C.°. The yield was 100 mg (83%); mp.: 148-153 C.°.

### EXAMPLE 5

### Preparation of 3-phenyl-4-(4-chlorosulfonyl-phenyl)-5-methyl-isoxazole (II)

6.65 g (0.1 mol) chlorosulfonic acid was dissolved in 50 ml of anhydrous dichloro methane. The solution was cooled to 0 C.° and solution of 4.7 g (0.02 mol) 3,4-diphenyl-5-methyl-isoxazole in 20 ml of anhydrous dichloro methane was added. The reaction mixture was stirred for 2 hours at room temperature and for another 10 hours at boiling point. The solvent was evaporated and the residue was poured onto 50 g of ice. This suspension was extracted twice with 40 ml of ethyl acetate. The combined organic phase was extracted 50 ml of water, dried over anhydrous magnesiumsulfate. After filtration and evaporation the residue was dissolved in hot cyclohexane and cooled to +15 C.° for crystallization. The precipitated product (4 g) was filtered and recrystallized from 50 ml of cyclohexane to afford the title compound (II) (3.7 g; mp.: 106-107 C.° ).

### EXAMPLE 6

### Preparation of solvate-free N-hydroxy-4-(3-phenyl-5-methyl-isoxazole-4-yl)-benzenesulfonamide

21.6 mg of N-hydroxy-4-(3-phenyl-5-methyl-isoxazole-4-yl)-benzene-sulfonamide monohydrate, prepared by example 1 was heated in vacuum (20 mbar) to 95 C.° in melt. Upon cooling to 25 C.° a glassy product is formed, melting range between 83-95 C.°, decomposition at 150 C.°. Purity: 99.8% (HPLC).

### EXAMPLE 7

### Tablet Containing N-hydroxy-4-(3-phenyl-5-methyl-isoxazole-4-yl)-benzene-sulfonamide monohydrate

10 mg of N-hydroxy-4-(3-phenyl-5-methyl-isoxazole-4-yl)-benzenesulfonamide monohydrate

2 mg of magnesium-stearate

4 mg of crospovidon

184 mg of microcrystallized cellulose

Total: 200 mg

N-hydroxy-4-(3-phenyl-5-methyl-isoxazole-4-yl)-benzenesulfonamide monohydrate and the components were mixed and formulated to tablet by compression.

### EXAMPLE 8

### Capsule containing N-hydroxy-4-(3-phenyl-5-methyl-isoxazole-4-yl)-benzene-sulfonamide monohydrate

10 mg N-hydroxy-4-(3-phenyl-5-methyl-isoxazole-4-yl)-benzenesulfonyl-amide monohydrate

10 mg ascorbic acid

The components were homogenized and filled into a capsule.

X-ray Diffraction Studies

The X-ray diffraction studies were carried out by Enraf-Nonius CAD4 diffractometer.

The ability of the N-hydroxy-4-(3-phenyl-5-methyl-isoxazole-4-yl)-benzene-sulfonamide that can form stoichiometric solid phase associates with different solvents. The best to report this feature are the crystallographic data. Further important characteristic that in all cases the N-hydroxy-4-(3-phenyl-5-methyl-isoxazole-4-yl)-benzene-sulfonamide, as the host molecule is binding the smaller (guest) molecules of solvents with hydrogen-bonds. For example, these binding are characterized by the crystal structure of the water-complex where the H-bonds are plotted with broken lines

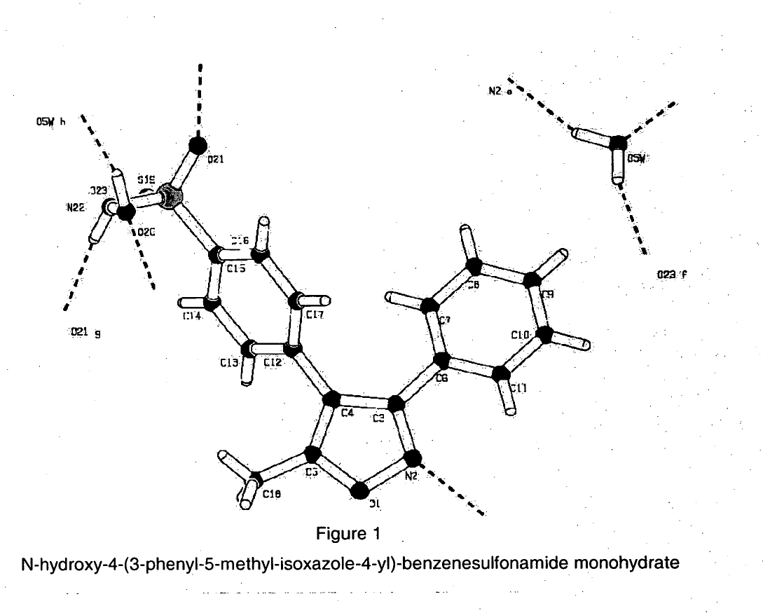

Hydrated inclusion of the N-hydroxy-4-(3-phenyl-5-methyl-isoxazole-4-yl)-benzene-sulfonamide (FIG. 1) is formed colourless, prismatic, monoclinic crystals. Space group: P2₁/c, cell constants at T=295(2) K temperature: a=7,659(1) Å, b=23.510(1) Å, c=9.148(1) Å, β=95.65(1)°, V=1639.2(3) Å³. The calculated density: Dₓ=1.412 Mg/m³. The sulfur atom is by the origo-dependent relative atomic coordinates of 0.23117(9) 0.27700(2) 0.52759(6) (x;y;z) with the σ error (between brackets) within the statistical significance of 3σ.

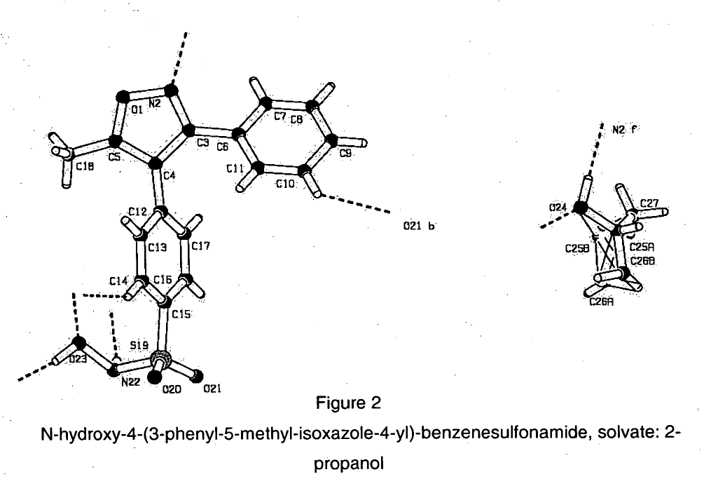

Complex formed with isopropanol in the ratio of 2:2 (FIG. 2) is characterized with the next data: colourless, prismatic, monoclinic crystals. Space group: P₁, cell constants at T=295(2) K temperature:•a =8.753(1) Å, b=10.858(1) Å, c=11.457(1) Å, α=70.47(1)°, β=79.83(1)°, γ=83.07(1)°, V=1007.9(2) Å³. The calculated density: Dₓ=1.287 Mg/m³. The sulfur atom is characterized by the origo-dependent relative atomic coordinates of 0.27950(4) 0.38112(3) 0.90833(3) (x;y;z) with the σ error (between brackets) within the statistical significance of 3σ.

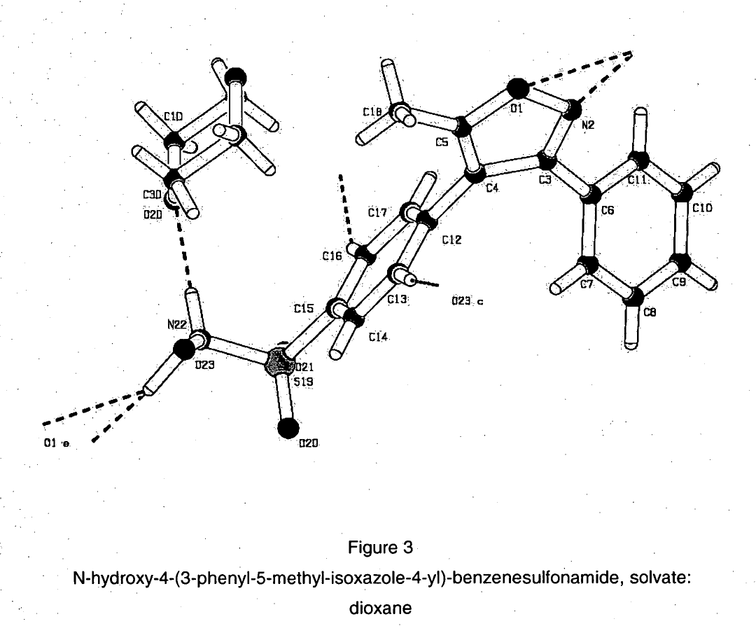

The dioxane inclusion (FIG. 3) is characterized with the following data: colourless, prismatic, monoclinic crystals. Space group: P2₁/c, cell constants at T=295(2) K temperature:•a =11.732(4) Å, b=10.171(7) Å, c=15.383(13) β=95.98(5)° V=1826(2) Å³. The calculated density: Dₓ=1.362 Mg/m³. The sulfur atom is characterized by the origo-dependent relative atomic coordinates of 0.60293(4); 0.31230(5); 0.78848(3) (x;y;z) with the σ error (between brackets) within the statistical significance of 3σ.

Powder diffraction curves calculated from cell constants and relative atomic coordinates of the above mentioned solid crystalline complexes are accorded with the measured. It is denotes the accordance of the crystals and the macroscopic samples.

1. N-hydroxy-4-(3-phenyl-5-methyl-isoxazole-4-yl)-benzenesulfonamide solvates of formula (I) 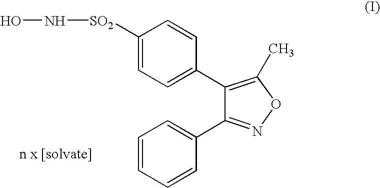
wherein n represents 0 or 1 mole,
[solvate] represents water, C₁-C₄ alcohol, C₁-C₄ alkylester of C₁-C₃ carboxylic acid or dioxane.

2. Compounds of formula (I) as claimed in claim 1, wherein n=1 and the solvate represents water.

3. Compounds of formula (I) as claimed in claim 1, wherein n=1 and the solvate represents ethylacetate.

4. Compounds of formula (I) as claimed in claim 1, wherein n=1 and the solvate represents 2-propanol.

5. Compounds of formula (I) as claimed in claim 1, wherein n=1 and the solvate represents dioxane.

6. Compound of formula (I) as claimed in claim 1, wherein n=0.

7. Mixture of compounds of formula (I) as claimed in claim 1, wherein n=1 and of compound of formula(I) as claimed in claim 1 wherein n=0.

8. Process for producing N-hydroxy-4-(3-phenyl-5-methyl-isoxazole-4-yl)-benzenesulfonamide solvates compounds of formula (I) wherein n=1 and solvate represents C₁-C₄ alkylester of C₁-C₃ carboxylic acid or dioxane, characterized by that the 3,4-diphenyl-5-methyl-izoxazole of formula (III) 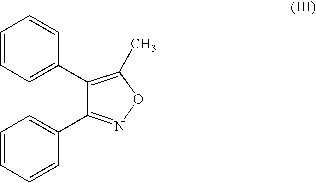
is reacted with chlorosulfonic acid and the product 3-phenyl-4-(4-chlorosulfonyl-phenyl)-5-methyl-isoxazole (II) 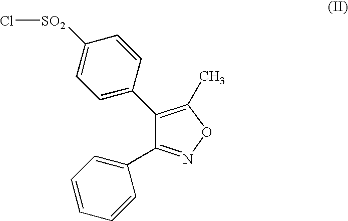
is reacted with hydroxylamine
a.) in mixture of water and water miscible solvent
b.) in mixture of non-water-miscible solvent and water in presence of phase transfer catalyst,
and the product is crystallized from a solvent chosen from a C₁-C₄ alkylester of C₁-C₃ carboxylic acid or dioxane.

9. Process as claimed in claim 8 characterized by that the phase-transfer catalyst is tetrabutylammonium hydrogensulfate.

10. Process as claimed in claim 8 characterized by that the recrystallization was carried out from ethyl acetate.

11. Process for producing N-hydroxy-4-(3-phenyl-5-methyl-isoxazole-4-yl)-benzenesulfonamide solvate compounds of formula (I) wherein n=1 and solvate represents water, characterized by that the 3,4-diphenyl-5-methyl-izoxazole of formula (III) 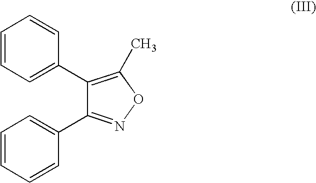
is reacted with chlorosulfonic acid and the product 3-phenyl-4-(4-chloro-sulfonyl-phenyl)-5-methyl-isoxazole (II) 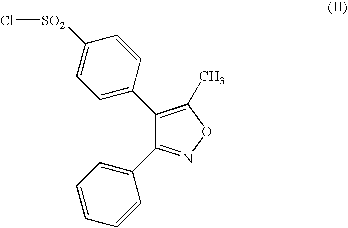
is reacted with hydroxylamine
a.) in mixture of water and water miscible solvent
b.) in mixture of non-water-miscible solvent and water in presence of phase transfer catalyst,
and the product is crystallized from a mixture of water and ethanol, optionally containing ascorbic acid.

12. Process for the preparation of compound of formula (I) wherein n=0 characterized by that the solvate is eliminated by heating from the compound of formula (I) wherein n=1.

13. Process for producing a mixture of compounds of formula (I) wherein n=1 and compound of formula (I) where n=0 in an optional ratio characterized by that the solvate can be removed in a required amount from the compounds of formula (I) where n=1 by heating and under reduced pressure.

14. Use of compounds of formula (I) claimed in any of claims 1-6 for producing pharmaceutical composition for treatment of osteoarthritis and rheumatoid arthritis and surgical and primary dysmenorrheal pains, based on anti-inflammatory and analgesic pharmacological model experiments.

15. Use of mixtures as claimed in claim 7 for producing pharmaceutical composition for treatment of osteoarthritis and rheumatoid arthritis and surgical and primary dysmenorrheal pains, based on anti-inflammatory and analgesic pharmacological model experiments.

16. Pharmaceutical composition containing a compound of formula (I) as claimed in any of claims 1-6 and one or more therapeutically acceptable pharmaceutical carriers.

17. Pharmaceutical composition containing a mixture as claimed in claim 7 and one or more therapeutically acceptable pharmaceutical carriers.

18. Pharmaceutical composition as claimed in claim 16 characterized by that the one of the carriers is ascorbic acid.

19. A method for treatment of osteoarthritis and rheumatoid arthritis and surgical and primary dysmenorrheal pains, based on anti-inflammatory and analgesic pharmacological model experiments, said method comprising treating the patient in need with therapeutically effective dose of a compound of formula (I) as claimed in any of claims 1-6.

20. A method for treatment of osteoarthritis and rheumatoid arthritis and surgical and primary dysmenorrheal pains, based on anti-inflammatory and analgesic pharmacological model experiments, said method comprising treating the patient in need with therapeutically effective dose of a mixture as claimed in claim 7.

21. A method for treatment of osteoarthritis and rheumatoid arthritis and surgical and primary dysmenorrheal pains, based on anti-inflammatory and analgesic pharmacological model experiments, said method comprising treating the patient in need with therapeutically effective dose of a pharmaceutical composition as claimed in any of claims 16-18.
